(19) United States
(12) Patent Application Publication
(10) Pub. No.: US 2012/0201764 A1
(43) Pub. Date: Aug. 9, 2012

(54) Natural Toothpaste

(76) Inventors: Douglas Arthur Day, Plymouth, MI (US); Elizabeth Irene Day, Brighton, MI (US)

(21) Appl. No.: 13/368,399

(22) Filed: Feb. 8, 2012

Related U.S. Application Data
(60) Provisional application No. 61/440,902, filed on Feb. 9, 2011.

(51) Int. Cl. A61K 8/60; A61Q 11/00; A61K 8/73; A61K 8/92; A61K 8/97

(52) U.S. Cl. 424/54; 424/49; 424/56; 424/58

(57) ABSTRACT

A natural toothpaste having a unique jelly-like composition and sweet flavor capable of providing sufficient oral hygiene care.

### CROSS REFERENCE TO RELATED APPLICATION

This application claims the benefit of U.S. Provisional Application Ser. No. 61/440,902 filed Feb. 9, 2011, entitled “Natural Children's Toothpaste,” the entire disclosure of the application being considered part of the disclosure of this application and hereby incorporated by reference.

### BACKGROUND OF THE INVENTION

1. Technical Field

The present invention is directed to an oral toothpaste and more specifically a natural toothpaste having an improved texture and taste.

2. Related Art

A large variety of oral care products exist on the market today of which almost all are predominately formed from synthetic ingredients. Concerns over the health effects of these synthetic ingredients have driven people to seek natural oral care products. To date, these natural oral products, in particular related to toothpaste, have not been able to achieve as pleasant of a feel in the mouth as compared to synthetic oral care products. In addition, for many natural toothpastes, the end product does not have a pleasant taste, have too limited of a shelf life, may not provide sufficient cleaning of the teeth when used in a manner similar to synthetic toothpaste and those natural toothpastes that provide sufficient cleaning are also substantially gritty in the mouth.

As parents increasingly worry about the types of products being ingested by children, more and more parents are attempting to use natural toothpaste for their children. One major issue with natural toothpaste for children has been the lack of a pleasant flavor and the gritty sensation caused by the cleaning agents in the natural toothpaste. Therefore, it has been difficult for many parents to convince their children to use natural toothpaste over artificial and synthetic toothpaste.

Natural toothpaste existed before the rise of synthetic or artificial toothpaste and a small group of people continued to use natural toothpaste throughout the years. One example of a basic natural toothpaste commonly used has been a slurry of baking soda. The major drawback to these natural toothpastes is that they typically do not perform satisfactory, have reduced performance when compared to the synthetic toothpaste. In addition to unsatisfactory performance including failure to provide adequate cleaning, limited shelf life, unpleasant taste and gritty sensation in the mouth, many of the natural toothpastes were overly abrasive creating long term negative effects on the consumer's teeth. In view of these issues, there currently is only a limited use of natural toothpaste, especially for children, even with the increased use of natural products by consumers.

In recent years, as consumers become more concerned about their health and the types of chemicals entering their bodies, natural toothpaste has experienced a small revival amongst a select group of consumers. However, to date, most consumers are dissatisfied with the overall performance of natural toothpastes. In addition, as discussed above, it is difficult to get children to regularly use natural toothpaste due to the consistency, taste and grittiness of the toothpaste. Therefore, there is a need for an oral care product, particularly a toothpaste that has a pleasant mouth feel, reduced grittiness, improved consistency and particularly for children.

### SUMMARY OF THE INVENTION

The present invention is directed to a natural toothpaste with improved performance characteristics. The natural toothpaste is generally formed out of water, a natural sweetener such as xylitol, vegetable glycerin, a foaming agent, a gelling agent or viscosity modifier such as carrageenan, a cleaning agent, such as calcium carbonate and organic flavors. The toothpaste is configured to have a pleasant mouth feel and a composition that is more jelly-like than ordinary toothpaste.

The present invention is generally directed to a natural oral care composition, specifically a toothpaste having a composition of 20-29% water; 18-24% xylitol; 37-45% vegetable glycerin; 1-2% foaming agent; 0.8-2.5% gelling agent; 9-13% abrasive; and 1-6% organic flavor. The above ranges are all by weight, as compared to the weight of the final composition. In addition, the ratio of water to vegetable glycerin is 0.5 to 0.75, preferably 0.6 to 0.7.

The composition is expected to preferably be equal to or greater than 24% by weight water, more preferably 26.5 and preferably equal to or less than 28% water. More specifically, in the illustrated examples, the water forms approximately 27.8% by weight.

The composition is expected to preferably have equal to or less than 40% by weight vegetable glycerin, more preferably equal to or less than 38% by weight vegetable glycerin. In addition, the composition is expected to preferably have equal to or more than 37.5% by weight vegetable glycerin, more preferably 38-40% by weight vegetable glycerin. In the illustrated examples, the vegetable glycerin is approximately 38% by weight.

The foaming agent is expected to be selected from the group consisting of: sodium coco sulfate, Quillaja, Yucca Saponins (all types), and sodium cocoyl glutamate. In the illustrated examples, the foaming agent is preferably sodium coco sulfate. The foaming agent preferably equal to or greater than 1% by weight, more preferably greater than or equal to approximately 1.1% by weight. In addition, the foaming agent is preferably less than or equal to approximately 1.7% by weight. More specifically, it is expected that the foaming agent is approximately 1.2-1.5% by weight, and in the illustrated examples is approximately 1.2% by weight.

The xylitol preferably forms greater than or equal to 20% by weight, more preferably is 20-22% by weight and in the specific examples is approximately 20% by weight. In addition, the xylitol preferably forms less than 23% by weight.

The gelling agent is preferably carrageenan and the carrageenan is preferably in the composition greater than 1% by weight. More preferably, the carrageenan forms greater than 1.1% by weight. In addition, preferably the carrageenan forms equal to or less than 2% by weight, more preferably equal to or less than 1.7% by weight. More specifically the carrageenan forms approximately 1-1.4% by weight and in the specific examples is approximately 1% by weight.

The abrasive is selected from the group consisting of calcium carbonate, mica and silica and is preferably calcium carbonate. The abrasive or calcium carbonate preferably forms greater than or equal to 10% by weight, and less than or equal to 12% by weight. In the illustrated examples, the calcium carbonate forms approximately 10% by weight of the final composition.

The organic flavor preferably forms greater than or equal to 2% by weight, and less than or equal to 4% by weight. It is expected that the organic flavor in the illustrated examples is best suited by approximately 2-3% by weight.

The present invention is also directed to a composition of wherein the water is 24-28% by weight, the xylitol is 20-22% by weight, the vegetable glycerin is 38-40% by weight, the foaming agent is 1.2-1.5% by weight, the gelling agent is carrageenan in the amount of 1.1-1.4% by weight, the abrasive is 10-12% by weight and the organic flavors are 2-3% by weight. The water has a ratio to vegetable glycerin of approximately 0.6 to 0.7. In addition, the composition may include a filler, which forms less than 5% by weight of the composition.

The present invention is also directed to a natural oral care composition, specifically toothpaste comprising: water to vegetable glycerin in ratio of 0.5 to 0.75; 18-24% xylitol by weight; 1-2.5% foaming agent; 0.8-2.5% carrageenan; 9-13% abrasive; and 1-6% organic flavor. All the ranges are weight percents of the total weight of the final composition, preferably has water of about 20-29% and the vegetable glycerin is about 37-45% by weight. More specifically, the water is approximately 24-28% by weight, the vegetable glycerin is 38-40% by weight, the xylitol is 20-22% by weight, the foaming agent is 1.201.5% by weight, the carrageenan is 1.1-1.4% by weight, the abrasive is 9-13% by weight and the organic flavor is 2-4% by weight. The composition may include filler in an amount less than 5% by weight.

The present invention is also directed to a natural oral care composition comprising: water to vegetable glycerin in ratio of 0.5 to 0.75; and having 19-29% by weight water, and 35-45% vegetable glycerin 17-24% xylitol by weight; 0.9-2.5% foaming agent; 0.7-2.5% carrageenan; 8.5-13% abrasive; 0.9-6% organic flavor; and an included filler up to 5% by weight.

### DETAILED DESCRIPTION OF THE PREFERRED EMBODIMENT

The natural toothpaste of the present invention is particularly characterized by how the specific range, given below, provides a unique consistency unlike other toothpastes while yet providing sufficient cleaning and oral hygiene, long shelf life, and not overly abrasive, while maintaining a pleasant mouth feel and flavor.

The toothpaste of the present invention generally includes about 20-29%, preferably 24-28% water and 37-44%, preferably 38-40% vegetable glycerin. Generally the ratio of water to glycerin would be about 0.5 to 0.75, and preferably about 0.6 to 0.7. The ratio of water to vegetable glycerin is generally important. If too much water relative to the amount of glycerin is present, the toothpaste generally would have a limited shelf life and be too watery for easy use by the consumers. The balance of vegetable glycerin to water within the ratios provided above for the present invention ensures a desired consistency and stability over time to the toothpaste.

In general, the water has an expected range, subject to the ranges of the other components of the toothpaste such as the above described ratio of water to glycerin of 20-29%, preferably 24-28% and more preferably, 26.5-28% by weight. In the illustrated examples discussed below, the water is 27.5-28% and more specifically having a targeted amount of approximately 27.8% by weight. The water is preferably, although other types of water could be used, including minerals. More specifically, to maintain the desired natural component of the toothpaste, the water used would not include fluoride, chlorine or other added chemicals common in most municipal water systems. The toothpaste includes an expected range, subject to the ranges of the other components, such as the above described ratio of water to glycerin of 37-45%, preferably 37.5-40%, more preferably 37.8-39% by weight. In the illustrated examples discussed below, the vegetable glycerin is 37.9-38.5% and more specifically having a target amount of approximately 38% by weight. The vegetable glycerin is preferably of a food grade, specifically such as a carbohydrate derived from coconut oil. The vegetable glycerin is capable of moistening and sweetening without raising glucose levels or contributing to tooth decay and acting as a natural preservative. The amount of vegetable glycerin could be slightly higher than the ranges but generally should not be less than the amount of the given range and the water could be slightly less than the amount of water given in the range but should not be higher than 29%, preferably 28%. Therefore, while the above ranges provide the best performance of the toothpaste, it is expected, but has not been proven, that water could go down to 20% and while it is expected, but has not been proven, the amount of vegetable glycerin could go up to 45%. Of course, if the ranges are outside the preferred range of 24-28% water and 38-40% vegetable glycerin, it is possible that the desired end consistency of the product may be negatively affected.

The composition also includes approximately 18-24% xylitol, preferably more than 19% and more preferably more than 20% xylitol by weight. In addition, the composition for the toothpaste includes preferably less than 23% and more preferably less than 22% xylitol by weight, and more preferably 20-22% xylitol. Xylitol is a naturally occurring sugar alcohol sweetener, more specifically a natural compound with a formula (CHOH)₃(CH₂OH)₂, and provides sweetness to the overall composition without the use of artificial sweeteners and without the use of sugars that negatively affect oral hygiene or are related to causing cavities, directly or indirectly. In addition, xylitol is a tooth-friendly, non-fermentable sugar alcohol and is specific in its inhibition of the mutans streptococci group of bacteria that is significant contributor to tooth decay. Xylitol inhibits mutans streptococci in the presence of other sugars with the exception of fructose and also inhibits the growth of streptococcus pneumoniae. The present invention uses xylitol as a sweetener for the toothpaste which is a natural sweetener with a pleasant mouth flavor and does not leave an after-taste commonly found with sorbitol. Xylitol also does not promote dental cavities. The addition of xylitol also has been found to be effective in reducing plaque accumulation.

The present invention also includes a natural foaming agent in the amount of approximately 1-2%, preferably 1.1-1.7% and more preferably 1.2-1.5% by weight of the composition of toothpaste. The foaming agent is selected from one or more natural foaming agents, preferably one of or a combination of sodium coco sulfate, quillaja, yucca saponins (all types) and sodium cocoyl glutamate. The examples in the tables below use sodium coco sulfate. The foaming agent is responsible in the present invention for the foam created as the person brushes their teeth. The range is especially selected in view of the other ingredients in the toothpaste such that sufficient foam but not too much foam is created. The alternative natural foaming agent to sodium coco sulfate, listed above may vary from the preferred 1.2-1.5% by weight used with sodium coco sulfate, but are expected to stay within the broad range of 1-2% by weight.

The present invention also includes a natural gelling agent, such as carrageenan in the amount of 0.8-2.5%, preferably 1-2% and more preferably 1.1-1.4% by weight to provide the desired consistency discussed above. The carrageenan is provided to create a more jelly-like effect and avoid the typical paste format.

The present invention also includes calcium carbonate in the amount of 10-12% as the cleaner or abrasive. Other natural abrasives may be substituted, such as various silicas or micas. The calcium carbonate of the present invention is especially selected to have a particle consistency of approximately 2-6 microns preferably 2.8-4.2 microns and more preferably 3-4 microns. Having a small micron particle size allows for the abrasion used for the cleaning of the teeth but also smoother feeling due to the fine particle size.

The present invention further may include USDA organic flavor or other natural flavors in the amounts of 1-6%, preferably 1.5-4% and more preferably 2-3% by weight. The organic flavors may be anything desired such as peppermint oils combined with other oils and other fruit extracts. Some organic flavors that have been found to work particularly well are those by Nature's Flavors® organic flavor concentrates which are concentrated water soluble liquids that contain no alcohol, specifically their mint, bubblegum, pink lemonade, lemonade, boysenberry, tangerine flavors with particular acceptance of the fruity flavors by children. As specified above, a portion of the present invention is to increase the desirability of natural toothpaste particularly with children that still provides sufficient cleaning and oral hygiene and would not be harmful if ingested, particularly to children. Therefore, the present invention also particularly is directed to some of the flavors listed above to, fruit flavors which are typically more attractive to children than mint flavors. Table 1 below provides ranges of the preferred composition of the toothpaste with the present invention. Of course, it should be recognized that a natural filler could be added in the amount of up to 5% by weight and the above ranges adjusted accordingly.

TABLE 1

| | | |
|---|---|---|
| 1 | Water | 20-29% |
| 2 | xylitol | 18-24% |
| 3 | Vegetable Glycerin | 37-45% |
| 4 | Foaming Agent | 1-2% |
| 5 | Carrageenan | 0.8-2.5% |
| 6 | Cleaner/Abrasive | 9-13% |
| 7 | USDA Organic Flavor | 1-6% |

Table 2 shows more preferable ranges for the toothpaste composition.

TABLE 2

| | | |
|---|---|---|
| 1 | Water | 24-28% |
| 2 | xylitol | 20-22% |
| 3 | Vegetable Glycerin | 38-40% |
| 4 | Sodium Coco Sulfate | 1.2-1.5% |
| 5 | Carrageenan | 1-1.4% |
| 6 | Calcium Carbonate | 10-12% |
| 7 | USDA Organic Flavor | 2-3% |

The composition provided for in Table 3 provides the best balance of mouth feel, flavor, cleaning, shelf life and desired end composition found by the inventors for the present invention.

TABLE 3

| | | | Batch | | Gal- | |
|---|---|---|---|---|---|---|
| Seq. | Compound | Portion | Size | Pounds | lons | Grams |
| 1 | Water | 27.8% | | 0.61 | 0.08 | 278.10 |
| 2 | xylitol | 20.0% | | 0.44 | 0.04 | 200.07 |
| 3 | Vegetable | 38.0% | | 0.84 | 0.07 | 380.13 |
| | Glycerin | | | | | |
| 4 | Foaming | 1.2% | | 0.03 | 0.01 | 12.00 |
| | Agent | | | | | |
| 5 | Carrageenan | 1.0% | 0.2255 | 0.02 | 0.01 | 10.00 |
| 6 | Abrasive/ | 10.0% | 2.2034 | 0.22 | 0.03 | 100.03 |
| | Cleaner | | | | | |
| 7 | USDA | 2.0% | | 0.04 | 0.01 | 20.01 |
| | Organic Flavor | | | | | |
| | TOTALS | 100.0000% | | 2.20 | 0.24 | 1000.35 |

In Table 3, the compositions of Table 2 are split up to form batches that separately mixed and then added together for the final composition. More specifically, the water in the amount of 27.8% in Table 3 is seen in Table 4 as coming in through two different process steps. The process steps in relation to Table 4 are discussed below.

TABLE 4

| | | | Batch | | Gal- | |
|---|---|---|---|---|---|---|
| Seq. | Compound | Portion | Size | Pounds | lons | Grams |
| 1A | Water | 19.5500% | | 0.43 | 0.06 | 195.57 |
| 2A | Sodium | 1.2000% | | 0.03 | 0.01 | 12.00 |
| | Coco Sulfate | | | | | |
| 1B | Water | 8.2500% | | 0.18 | 0.02 | 82.53 |
| 2B | Xylitol | 20.0000% | | 0.44 | 0.04 | 200.07 |
| 3B | Vegetable | 38.0000% | | 0.84 | 0.10 | 380.13 |
| | Glycerin | | | | | |
| 4B | Carrageenan | 1.0000% | 0.2255 | 0.02 | 0.01 | 10.00 |
| 5 | Calcium | 10.0000% | 0.0000 | 0.22 | 0.03 | 100.03 |
| | Carbonate | | | | | |
| 6 | USDA | 2.0000% | | 0.04 | 0.01 | 20.01 |
| | Organic Flavor | | | | | |
| | Totals | 100.0000% | | 2.02 | 0.27 | 1000.35 |

To prepare the toothpaste composition, the water (1A) of 19.55% in Table 4 is combined with the foaming agent, sodium coco sulfate (2A) of 1.200% by weight of the final composition. The water (1A) and sodium coco sulfate (2A) are generally heated to 85° C. and mixed together. Separately, the carrageenan (4B) of 1.00% by weight of the final mixture is added together with vegetable glycerin (3B) of 38.06% by weight of the final mixture. The carrageenan (4B) and vegetable glycerin (3B) are stirred generally for five minutes and then the xylitol (2B) having 20.00% by weight of the final mixture is added to the mixture along with the second source of water (1B) forming a 8.25% by weight of the final composition. The water (1B), xylitol (2B), vegetable glycerin (3B), and carrageenan (4B) are then heated to 85° C. and mixed sufficiently together. Once sufficiently mixed, they are added to the mixture of water (1A) and sodium coco sulfate (2A) and then the calcium carbonate (5) in the amount of 10.00% by weight of the final mixture and USDA organic flavor (6) forming 2% by weight of the final composition are also added. The composition is then further mixed to its final formulation. Of course, variations on the method steps provided above may occur without effecting the end composition of the toothpaste.

1. A natural oral care composition comprising:
20-29% water by weight;
18-24% xylitol by weight;
37-45% vegetable glycerin by weight;
1-2% foaming agent by weight;
0.8-2.5% gelling agent by weight;
9-13% abrasive by weight; and
1-6% organic flavor by weight.

2. The composition of claim 1 wherein the ratio of water to vegetable glycerin is 0.5 to 0.75.

3. The composition of claim 1 wherein the ratio of water to vegetable glycerin is 0.6 to 0.7.

4. The composition of claim 1 having at least 24% by weight water.

5. The composition of claim 4 having at least 26.5% by weight water.

6. The composition of claim 4 having equal to or less than 28% water.

7. The composition of claim 1 wherein said water forms approximately 27.8% by weight.

8. The composition of claim 1 wherein said vegetable glycerin is less than or equal to 40% by weight.

9. The composition of claim 8 wherein said vegetable glycerin is less than or equal to 39% by weight.

10. The composition of claim 1 wherein said vegetable glycerin is more than 37.5% by weight.

11. The composition of claim 10 wherein said vegetable glycerin is 38-40% by weight.

12. The composition of claim 1 wherein said vegetable glycerin is approximately 38% by weight.

13. The composition of claim 1 wherein said foaming agent is selected from the group consisting of: sodium coco sulfate, Quillaja, Yucca Saponins (all types), and sodium cocoyl glutamate.

14. The composition of claim 13 wherein said foaming agent is sodium coco sulfate.

15. The composition of claim 1 wherein said foaming agent is approximately equal to or greater than 1% by weight.

16. The composition of claim 15 wherein said foaming agent is greater than or equal to approximately 1.1% by weight.

17. The composition of claim 16 wherein said foaming agent is less than or equal to approximately 1.7% by weight.

18. The composition of claim 1 wherein said foaming agent is approximately 1.2-1.5% by weight.

19. The composition of claim 1 wherein said foaming agent is approximately 1.2% by weight.

20. The composition of claim 1 wherein said xylitol forms greater than or equal to 20% by weight.

21. The composition of claim 21 wherein said xylitol forms approximately 20-22% by weight.

22. The composition of claim 21 wherein said xylitol forms approximately 20% by weight.

23. The composition of claim 1 wherein said xylitol forms less than 23% by weight.

24. The composition of claim 1 wherein said gelling agent is carrageenan.

25. The composition of claim 24 wherein said carrageenan forms greater than or equal to 1% by weight.

26. The composition of claim 25 wherein said carrageenan forms greater than or equal to 1.1% by weight.

27. The composition of claim 24 wherein said carrageenan forms less than or equal to 2% by weight.

28. The composition of claim 24 wherein said carrageenan forms less than or equal to 1.7% by weight.

29. The composition of claim 24 wherein said carrageenan forms approximately 1-1.4% by weight.

30. The composition of claim 24 wherein said carrageenan forms approximately 1% by weight.

31. The composition of claim 1 wherein said abrasive is selected from the group consisting of calcium carbonate, mica and silica.

32. The composition of claim 31 wherein said abrasive is calcium carbonate.

33. The composition of claim 32 wherein said calcium carbonate forms greater than or equal to 10% by weight.

34. The composition of claim 33 wherein said calcium carbonate forms less than or equal to 12% by weight.

35. The composition of claim 1 wherein said abrasive is calcium carbonate in the amount of approximately 10% by weight.

36. The composition of claim 1 wherein said organic flavor forms greater than or equal to 2% by weight.

37. The composition of claim 1 wherein said organic flavor forms less than or equal to 4% by weight.

38. The composition of claim 1 having organic flavor in the amount of approximately 2-3% by weight.

39. The composition of claim 1 wherein said water is 24-28% by weight, said xylitol is 20-22% by weight, said vegetable glycerin is 38-40% by weight, said foaming agent is 1.2-1.5% by weight, said gelling agent is carrageenan in the amount of 1.1-1.4% by weight, said abrasive is 10-12% by weight and said organic flavors are 2-3% by weight.

40. The composition of claim 39 wherein said water has a ratio to vegetable glycerin of approximately 0.6 to 0.7.

41. The composition of claim 1 further including a filler, and wherein said filler forms less than 5% by weight.

42. A natural oral care composition comprising:
water to vegetable glycerin in ratio of 0.5 to 0.75;
18-24% % xylitol by weight;
1-2.5% foaming agent;
0.8-2.5% carrageenan;
9-13% abrasive; and
1-6% organic flavor.

43. The composition of claim 42 wherein said water is 20-29% and said vegetable glycerin is 37-45% by weight.

44. The composition of claim 42 wherein said water is approximately 24-28% by weight, said vegetable glycerin is 38-40% by weight, said xylitol is 20-22% by weight, said foaming agent is 1.2-1.5% by weight, said carrageenan is 1.1-1.4% by weight, said abrasive is 9-13% by weight and said organic flavor is 2-4% by weight.

45. The composition of claim 42 including a filler in an amount less than 5% by weight.

46. A natural oral care composition comprising:
water to vegetable glycerin in ratio of 0.5 to 0.75; and having 19-29% by weight water, and 35-45% vegetable glycerin
17-24% % xylitol by weight;
0.9-2.5% foaming agent;
0.7-2.5% carrageenan;
8.5-13% abrasive;
0.9-6% organic flavor; and
an included filler up to 5% by weight.
